(19) United States
(12) Patent Application Publication
(10) Pub. No.: US 2004/0248970 A1
(43) Pub. Date: Dec. 9, 2004

(54) CBD-delta8-THC composition

(76) Inventors: G.R. Barrie Webster, Manitoba (CA); Leonard P. Sarnia, Manitoba (CA)

Correspondence Address: ADE & COMPANY, 1700-360 MAIN STREET, WINNIPEG, MB, R3C3Z3, CA

(21) Appl. No.: 10/820,223

(22) Filed: Apr. 8, 2004

Related U.S. Application Data
(60) Provisional application No. 60/461,575, filed on Apr. 10, 2003.

(51) Int. Cl. A61K 31/353

(52) U.S. Cl. 514/454

(57) ABSTRACT

An anti-emetic composition comprising Δ⁸-tetrahydrocannabinol and cannabidiol and the use thereof is described.

### PRIOR APPLICATION INFORMATION

The instant application claims priority on 60/461,575, filed Apr. 10, 2003.

### FIELD OF THE INVENTION

The present invention relates generally to the field of pharmaceutical compositions. More specifically, the present invention relates to a pharmaceutical composition comprising CBD and Δ⁸-THC.

### BACKGROUND OF THE INVENTION

Recently, public interest in Cannabis as medicine has been growing, based in no small part on the fact that Cannabis has long been considered to have medicinal properties, ranging from treatment of cramps, migraines, convulsions, appetite stimulation and attenuation of nausea and vomiting. In fact, a report issued by the National Academy of Sciences' Institute of Medicine indicated that the active components of Cannabis appear to be useful in treating pain, nausea, AIDS-related weight loss or “wasting”, muscle spasms in multiple sclerosis as well as other problems. Advocates of medical marijuana argue that it is also useful for glaucoma, Parkinson's disease, Huntington's disease, migraines, epilepsy and Alzheimers disease.

Marijuana refers to varieties of Cannabis having a high content of Δ⁹-tetrahydrocannabinol (Δ⁹-THC), which is the psychoactive ingredient of marijuana whereas industrial hemp refers to varieties of the Cannabis plant that have a low content of Δ⁹-THC.

The controversy regarding the medicinal use of marijuana is centered not only on what is delivered but on how it is delivered. Specifically, the primary method used to deliver marijuana into a patient's system is by smoking the marijuana; however, smoking increases an individual's risk for cancer, lung damage and emphysema. Furthermore, as discussed above, marijuana does contain high levels of a psychoactive drug, Δ⁹-THC. As such, there has been considerable debate as to whether or not the potential health benefits of smoking marijuana outweigh the health risks. In addition, the psychoactive activity of Δ⁹-THC has led to reluctance of public acceptance of medicines including this compound.

However, studies have revealed that the activity in animals of several samples of marijuana differed significantly, differences which could not be attributed solely to Δ⁹-THC content (Carlini et al, 1970, Psychopharmacologia 18: 82; Karniol and Carlini, 1972, J Pharm Pharmacol 24: 833). This led to the hypothesis that other cannabinoid compounds were interfering with Δ⁹-THC's effects. Specifically, it was shown that CBD was able to block the excitatory effects of Δ⁹-THC and to potentate the depressant effects of Δ⁹-THC (Karniol and Carlini, 1973, Psychopharmacologia 33: 53) while CBD, administered on its own, had no noticeable effects (Mincis et al, 1973, Rev Ass Med Brasil 19: 185). In a further study, Karniol et al (1974, Eur J Pharma 28: 172-177) showed that dosages of 15, 30 and 60 mg CBD in admixture with 30 mg Δ⁹-THC (in orange juice) attenuated several effects of Δ⁹-THC compared to controls, such as pulse rate acceleration, time production impairment and psychological disturbances. As will be apparent, this corresponds to a CBD:Δ⁹-THC ratio of between 0.5:1 to 2:1. Dalton et al (1976, Clin Pharmacol Ther 19: 300-309) observed attenuation of the Δ⁹-THC effects when both CBD and Δ⁹-THC were inhaled simultaneously, at 10.5 mg and 1.7 mg respectively (CBD:Δ⁹-THC ratio of 6:1), but detected no interaction with the pretreatment of CBD. It is important to note that there is also evidence that heating leads to conversion of CBD into Δ⁹-THC (Mikes and Waser, 1971, Science 172: 1158), meaning that the accuracy of these results must be questioned due to the delivery method used. Zuardi et al (1982, Psychopharmacology 76: 245-250) administered 35 mg Δ⁹-THC and 70 mg CBD in lemon juice (CBD:Δ⁹-THC ratio of 2:1) to volunteers and observed that the anxiety effect associated with Δ⁹-THC was lessened by CBD but that the tachycardia associated with Δ⁹-THC was not affected. Based on this result, the authors propose that CBD and Δ⁹-THC have independent and opposing psychometric effects on man. It is however important to note that the psychometric effects were measured using a “self-rating scale’.

It is also of note that a study by Hollister and Gillespie (1975, Clin Pharmacol Ther 18: 80-83) did not observe any effect between CBD (40 mg) and Δ⁹-THC (20 mg) when administered orally, except of retarding and prolonging the duration of the Δ⁹-THC effect.

It is important to note that the above-described studies were focused on moderating the psychoactive effects of Δ⁹-THC and did not examine or consider the effect of CBD on other Δ⁹-THC effects, such as Δ⁹-THC's anti-emetic properties. It is also of note that it has been suggested that Δ⁹-THC has limited use as an anti-emetic drug, particularly in cancer therapy, due to the side effects associated withΔ⁹-THC, including psychological high, anxiety, hypotension and sedation (Mechoulam and Feigenbaum, 1987, Prog Medicinal Chem 24:159-207).

Furthermore, Δ⁹-THC is only one of a family of about 60 bi- and tri-cyclic compounds named cannabinoids. For example, Δ⁸-THC is a double bond isomer of Δ⁹-THC and is a minor constituent of most varieties of Cannabis (Hollister and Gillespie, 1972, Clin Pharmacol Ther 14: 353). The major chemical difference between the two compounds is that Δ⁹-THC is easily oxidized to cannabinol whereas Δ⁸-THC does not and is in fact very stable. Δ⁸-THC, for the most part, produces similar psychometric effects as does Δ⁹-THC, but is generally considered to be 50% less potent than Δ⁹-THC and has been shown in some cases to be 3-10 times less potent. Δ⁸-THC has also been shown to be more (200%) effective an anti-emetic than Δ⁹-THC and has been used as an anti-emetic in children, based on the belief that the side effects of Δ⁹-THC and Δ⁸-THC, such as anxiety and dysphoria, are more prevalent in adults than children (Abrahamov et al, 1995, Life Sciences 56: 20972102). It is also of note that the effect of CBD on Δ⁸-THC has not been investigated.

### SUMMARY OF THE INVENTION

According to a first aspect of the invention, there is provided a pharmaceutical composition for use as an anti-emetic comprising an effective amount of Δ⁸-tetrahydrocannabinol and cannabidiol.

The pharmaceutical composition may comprise 2-10 parts Δ⁸-tetrahydrocannabinol to 1 part cannabidiol.

The pharmaceutical composition may comprise 2-40 mg Δ⁸-tetrahydrocannabinol and 0.2-20 mg cannabidiol.

The pharmaceutical composition may comprise 2-10 mg Δ⁸-tetrahydrocannabinol and 0.2-5 mg cannabidiol.

According to a second aspect of the invention, there is provided a method of ameliorating vomiting or nausea in an individual in need of such treatment comprising:

providing a pharmaceutical composition comprising Δ⁸-tetrahydrocannabinol and cannabidiol; and

administering an effective amount of said composition to the individual.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

### DEFINITIONS

As used herein, “purified” does not require absolute purity but is instead intended as a relative definition. For example, purification of starting material or natural material to at least one order of magnitude, preferably two or three orders of magnitude is expressly contemplated as falling within the definition of “purified”.

As used herein, the term “isolated” requires that the material be removed from its original environment.

As used herein, the term “treating” in its various grammatical forms refers to preventing, curing, reversing, attenuating, alleviating, minimizing, suppressing or halting the deleterious effects of a disease state, disease progression, disease causitive agent other abnormal condition.

As used herein, “anti-emetic’ refers to compounds capable of reducing nausea, enhancing appetite and/or reducing vomiting in an individual.

As used herein, “Δ⁸-THC” refers to Δ⁸-tetrahydrocannabinol.

As used herein, “CBD” refers to cannabidiol.

As used herein, “effective amount” refers to the administration of an amount of a given compound that achieves the desired effect. For example, regarding the combination of CBD and Δ⁸-THC, an “effective amount” is an amount sufficient for or that is capable of reducing nausea or vomiting and/or enhancing appetite in a patient or individual in need of such treatment. The patient may be a human patient.

Described herein is the preparation and use of a novel pharmaceutical composition comprising CBD and Δ⁸-THC. In an exemplary use, the composition is used as an anti-emetic. Specifically, the pharmaceutical composition is prepared by mixing isolated, purified or synthetic CBD with isolated, purified or synthetic Δ⁸-THC at a ratio of 2-10 parts Δ⁸-THC to 1 part CBD. As will be apparent to one knowledgeable in the art, the specific dosage may vary according to the condition, age and/or weight as well as other factors relating to the general health of the patient. However, in one embodiment, the pharmaceutical combination may comprise 2-40 mg Δ⁸-THC and 0.2-20 mg CBD. In an alternative embodiment, the pharmaceutical combination may comprise 2-10 mg Δ⁸-THC and 0.2-5.0 mg CBD. As will be appreciated by one of skill in the art, the total amount in milligrams of each component will vary according to the size of the pharmaceutical composition, which may be for example in a pill, tablet, capsule, tincture or liquid form.

In some embodiments, the chemicals are purified and blended together to produce a formulation similar in form to that for Marinol®. In these formulations, the active ingredient is dissolved in sesame seed oil or a similar oil and enclosed in a gel-capsule. In other embodiments, the formulation may be arranged to be used as an injectable or as an aerosol. In these embodiments, as will be apparent to one of skill in the art, the appropriate pharmaceutically-acceptable additives may be added so that the pharmaceutical composition is in the appropriate form.

As will be appreciated by one knowledgeable in the art, the formulation may be used as, for example, an anti-emetic, appetite stimulant, or as a treatment for nausea, dementia, Alzheimer's disease, glaucoma, high blood pressure, inflammation or multiple sclerosis. As such, when administered to an individual in need of such treatment, the pharmaceutical composition of Δ8-THC and CBD will accomplish at least one of the following: reduce nausea, promote or stimulate appetite, reduce vomiting and/or promote a general feeling of well-being.

In use, the pharmaceutical composition is administered to a patient suffering from vomiting or nausea or at risk of developing these symptoms, possibly due to another treatment. As discussed above, Δ⁸-THC is a potent anti-emetic but has the side effect of also being psychoactive. However, combining Δ⁸-THC with CBD diminishes these psychoactive effects, resulting in an anti-emetic with no or lessened psychoactive side effects.

In some embodiments, the pharmaceutical composition may be combined with other compounds or compositions known in the art such that the pharmaceutical composition is in the form of, for example, a pill, tablet, capsule or liquid form. The pharmaceutical composition may also be arranged to be injected, taken orally as a liquid or be in an aerosol form.

It is of note that the pharmaceutical composition discussed above may be prepared to be administered in a variety of ways, for example orally or intravenously, using means known in the art and as discussed below. In other embodiments, the pharmaceutical composition may be administered as a patch.

In some embodiments, the pharmaceutical composition at concentrations or dosages discussed herein may be combined with a pharmaceutically or pharmacologically acceptable carrier, binder, excipient or diluent, either biodegradable or non-biodegradable. See, for example, Remington: The Science and Practice of Pharmacy, 1995, Gennaro ed.

While the preferred embodiments of the invention have been described above, it will be recognized and understood that various modifications may be made therein, and the appended claims are intended to cover all such modifications which may fall within the spirit and scope of the invention.

1. A pharmaceutical composition for use as an anti-emetic comprising an effective amount of Δ⁸-tetrahydrocannabinol and cannabidiol.

2. The pharmaceutical composition according to claim 1 comprising 2-10 parts Δ⁸-tetrahydrocannabinol to 1 part cannabidiol.

3. The pharmaceutical composition according to claim 1 comprising 2-40 mg Δ⁸-tetrahydrocannabinol and 0.2-20 mg cannabidiol.

4. The pharmaceutical composition according to claim 1 comprising 2-10 mg Δ⁸-tetrahydrocannabinol and 0.2-5 mg cannabidiol.

5. A method of ameliorating vomiting or nausea comprising:
providing a pharmaceutical composition comprising Δ⁸-tetrahydrocannabinol and cannabidiol; and
administering an effective amount of said composition to a patient.

6. The method according to claim 5 wherein the pharmaceutical composition comprises 2-10 parts Δ⁸-tetrahydrocannabinol to 1 part cannabidiol.

7. The method according to claim 5 wherein the pharmaceutical composition comprises 2-40 mg Δ⁸-tetrahydrocannabinol and 0.2-20 mg cannabidiol.

8. The method according to claim 5 wherein the pharmaceutical composition comprises 2-10 mg Δ⁸-tetrahydrocannabinol and 0.2-5 mg cannabidiol.
